(19) United States
(12) Patent Application Publication
(10) Pub. No.: US 2026/0130779 A1
(43) Pub. Date: May 14, 2026

(54) SYSTEMS AND METHODS FOR STENT PLACEMENT

(71) Applicant: BOSTON SCIENTIFIC SCIMED, INC., Maple Grove, MN (US)

(72) Inventors: Peter L. Dayton, Brookline, MA (US); Raymond David Gessler, III, Roberts, WI (US); Mark Phillip Olson, Saint Anthony, MN (US); Aaron Abbott, Columbia Heights, MN (US); Adam Skelton, Natick, MA (US)

(73) Assignee: BOSTON SCIENTIFIC SCIMED, INC., Maple Grove, MN (US)

(21) Appl. No.: 19/386,815

(22) Filed: Nov. 12, 2025

Related U.S. Application Data
(60) Provisional application No. 63/719,714, filed on Nov. 13, 2024.

(51) Int. Cl. A61F 2/966; A61B 18/00; A61F 2/82; A61F 2/95

(52) U.S. Cl. CPC A61F 2/966; A61B 18/00; A61F 2/82; A61F 2/9517; A61B 2018/00077; A61B 2018/00083; A61B 2018/00178; A61B 2018/00184; A61B 2018/00595

(57) ABSTRACT

Medical device systems are disclosed. A system for positioning a stent within a tissue pathway includes a handle, an inner shaft having a lumen, a proximal end coupled to the handle and a distal end coupled to a tip member. The system also includes an outer shaft having a proximal end region and a lumen, wherein at least a portion of the inner shaft extends within the lumen of the outer shaft. The system also includes a grip member coupled to the outer shaft, a stent positioned between an inner surface of the outer shaft and an outer surface of the inner shaft, an electrically conductive element coupled to the tip member, an electrical wire coupled to the handle and the electrically conductive element and a cannula assembly configured to be positioned within the lumen of the inner shaft, the cannula assembly including an electrode and an insulation member.

### CROSS-REFERENCE TO RELATED APPLICATIONS

The application claims the benefit of U.S. Provisional Patent Application Ser. No. 63/719,714, filed on Nov. 13, 2024, the disclosure of which is incorporated herein by reference.

### TECHNICAL FIELD

This application relates generally to medical methods and devices. More specifically, the present disclosure relates to stents and methods for their use in establishing drainage pathways with medical procedures.

### BACKGROUND

A wide variety of medical devices have been developed for medical use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. A system for positioning a stent within a tissue pathway includes a handle, an inner shaft having a lumen, a proximal end coupled to the handle and a distal end coupled to a tip member. The system also includes an outer shaft having a proximal end region and a lumen, wherein at least a portion of the inner shaft extends within the lumen of the outer shaft. The system also includes a grip member coupled to the proximal end region of the outer shaft, a stent positioned between an inner surface of the outer shaft and an outer surface of the inner shaft, an electrically conductive element coupled to the tip member, an electrical wire having a proximal end coupled to the handle and a distal end coupled to the electrically conductive element and a cannula assembly configured to be positioned within the lumen of the inner shaft, wherein the cannula assembly includes an electrode and an insulation member.

Alternatively or additionally to any of the embodiments above, wherein the cannula assembly is configured to translate relative to the tip member.

Alternatively or additionally to any of the embodiments above, wherein the tip member includes a lumen in fluid communication with the lumen of the inner shaft, and wherein the cannula assembly is configured to translate within both the lumen of the inner shaft and the lumen of the tip member.

Alternatively or additionally to any of the embodiments above, wherein the cannula assembly is configured to translate relative to the lumen of the inner shaft and the lumen of the tip member such that the electrode is positioned distal of the distal end of the tip member.

Alternatively or additionally to any of the embodiments above, wherein the electrode is configured to cauterize tissue when positioned distal of the distal end of the tip member.

Alternatively or additionally to any of the embodiments above, wherein the electrode is configured to be retracted into the lumen of the tip member, the lumen of the inner shaft or both the lumen of the tip member and the lumen of the inner shaft after cauterizing tissue.

Alternatively or additionally to any of the embodiments above, wherein the electrode includes a lumen, and wherein the electrode lumen is configured to permit a guidewire to extend therein.

Alternatively or additionally to any of the embodiments above, wherein the electrically conductive element is configured to cauterize tissue along a tissue pathway formed by the electrode.

Alternatively or additionally to any of the embodiments above, wherein the handle includes an actuation member configured to translate the cannula assembly within the lumen of the inner shaft.

Alternatively or additionally to any of the embodiments above, wherein the insulation member is fixedly attached to the electrode.

Alternatively or additionally to any of the embodiments above, wherein the insulation member is slidable relative to the electrode.

Alternatively or additionally to any of the embodiments above, wherein the insulation member is configured to shift between a first position in which a distal end of the insulation member is positioned proximal to the distal end of the electrode to a second position in which the distal end of the insulation member is positioned distal to the distal end of the electrode.

Alternatively or additionally to any of the embodiments above, wherein the insulation member covers the distal end of the electrode in the second configuration.

Alternatively or additionally to any of the embodiments above, wherein a guidewire is configured to translate through a lumen of the electrode when the insulation member is covering the distal end of the electrode.

Another example system for positioning a drainage stent between a biliary duct and the stomach includes a handle, an inner shaft having a lumen, a proximal end coupled to the handle and a distal end coupled to a tip member. The system also includes an outer shaft having a proximal end region and a lumen, wherein at least a portion of the inner shaft extends within the lumen of the outer shaft. The system also includes a grip member coupled to the proximal end region of the outer shaft. The system also includes a stent positioned between an inner surface of the outer shaft and an outer surface of the inner shaft. The system also includes an electrically conductive element coupled to the tip member and an electrical wire having a proximal end coupled to the handle and a distal end coupled to the electrically conductive element. Further, the electrically conductive element is configured to cauterize a tissue pathway between the biliary duct and the stomach such that the stent can be deployed within a portion of the tissue pathway contiguous in time with forming the tissue pathway in order to reduce leakage.

Alternatively or additionally to any of the embodiments above, further comprising a cannula assembly configured to translate relative to the tip member.

Alternatively or additionally to any of the embodiments above, wherein the tip member includes a lumen in fluid communication with the lumen of the inner shaft, and wherein the cannula assembly is configured to translate within both the lumen of the inner shaft and the lumen of the tip member.

Alternatively or additionally to any of the embodiments above, wherein the cannula assembly is configured to translate within both the lumen of the inner shaft and the lumen of the tip member such that the electrode is positioned distal of the distal end of the tip member.

Alternatively or additionally to any of the embodiments above, wherein the electrode is configured cauterize tissue when positioned distal of the distal end of the tip member.

A method for positioning a drainage stent between a biliary duct and the stomach includes positioning a stent delivery system adjacent a wall of the stomach, the stent delivery system including a handle, an inner shaft having a lumen, a proximal end coupled to the handle and a distal end coupled to a tip member. The system also includes an outer shaft having a proximal end region and a lumen, wherein at least a portion of the inner shaft extends within the lumen of the outer shaft. The system also includes a grip member coupled to the proximal end region of the outer shaft. The system also includes a stent positioned between an inner surface of the outer shaft and an outer surface of the inner shaft. The system also includes an electrically conductive element coupled to the tip member and an electrical wire having a proximal end coupled to the handle and a distal end coupled to the electrically conductive element. The system also includes a cannula assembly configured to be positioned within the lumen of the inner shaft, wherein the cannula assembly includes an electrode and an insulation member. The method also includes advancing the cannula assembly within the lumen of the inner shaft to a position in which a distal end of the electrode is distal to the tip member, cauterizing tissue with the electrode to form a pathway between the biliary duct and the stomach and retracting the cannula assembly into the lumen of the inner shaft.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example system for accessing the stomach and/or biliary tract.

FIG. 2 is a partial cross-sectional view of a portion of the system of FIG. 1 in a first configuration.

FIG. 3 is a partial cross-sectional view of a portion of the system of FIG. 1 in a second configuration.

FIG. 4 schematically depicts an access needle positioned within a portion of the digestive system.

FIG. 5 schematically depicts a guidewire extending through the access needle positioned within a portion of the digestive system.

FIG. 6 schematically depicts a portion of the system of FIG. 1 positioned within a portion of the digestive system.

FIGS. 7-10 illustrate a method for deploying a stent within a portion of the digestive system.

FIG. 11 is a partial cross-sectional view of a portion of an example system for accessing the stomach and/or biliary tract in a first configuration.

FIG. 12 is a partial cross-sectional view of a portion of an example system for accessing the stomach and/or biliary tract in a second configuration.

FIGS. 13-14 illustrate a portion of the example system for accessing the stomach and/or biliary tract shifting between a first configuration and a second configuration.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Biliary obstruction may commonly be caused by a variety of conditions such as pancreatic cancer, cholangiocarcinoma, or metastatic tumors compressing the bile ducts. Different approaches may be utilized for managing biliary obstruction, such as endoscopic retrograde cholangiopancreatography or percutaneous transhepatic biliary drainage. However, when such approaches are not feasible or have failed, alternative medical procedures may be utilized. For example, endoscopic ultrasound-guided hepaticogastrostomy (EUS-HGS) is a minimally invasive procedure that establishes a drainage pathway between the bile duct and the stomach to bypass a biliary obstruction.

An endoscopic ultrasound-guided hepaticogastrostomy procedure may require precise placement of a drainage stent between a duct (e.g., biliary duct, hepatic duct, bile duct, etc.) and the stomach. The procedure may include entering the stomach through a first organ or structure, such as the esophagus. Once positioned in the stomach, a medical device may be utilized to create a fistula between the stomach and the duct (e.g., biliary duct, hepatic duct, bile duct, etc.), thereby forming a pathway for placement of a stent (e.g., a covered stent) configured to drain fluid from the duct (e.g., biliary duct, hepatic duct, bile duct, etc.) into the stomach. The fluid may then exit the body via the large and small intestines.

When deploying a drainage stent between adjacent body lumens, organs, or other structures, it is typically necessary to penetrate both a wall of the first body organ (e.g., stomach) through which access is established and a wall of a second body lumen (e.g., biliary duct, hepatic duct, bile duct, etc.). When initially forming such access penetrations, there may be a risk of leakage from either or both of the access body organ and the target body lumen into the surrounding space. In some procedures, such as those involving endoscopic ultrasound-guided hepaticogastrostomy, loss of body fluid into surrounding tissues and body cavities can present a substantial risk to the patient. The risk can be exacerbated when it is necessary to not only penetrate the stomach wall to gain initial access (e.g., with a needle), but to when forming the fistula prior to placement of the stent.

Thus, it would be desirable to establish an initial stomach wall penetration and fistula formation between the stomach and duct (e.g., biliary duct, hepatic duct, bile duct, etc.) in order to deploy a drainage stent while minimizing the risk of body fluid leakage. It is also desirable to minimize trauma and damage to the tissue surrounding the stomach wall and duct (e.g., biliary duct, hepatic duct, bile duct, etc.) during placement of the stent. It would be further desirable to provide improved medical devices and methods which are capable of being deployed from an endoscope present in the first body organ (e.g., stomach) to access adjacent body lumens or cavities (e.g., biliary duct, hepatic duct, bile duct, etc.) while minimizing the risk of leakage. Medical devices and methods using such medical devices that are configured to be deployed from an endoscope present in a first body organ to access adjacent body lumens or cavities while minimizing the risk of leakage are disclosed herein.

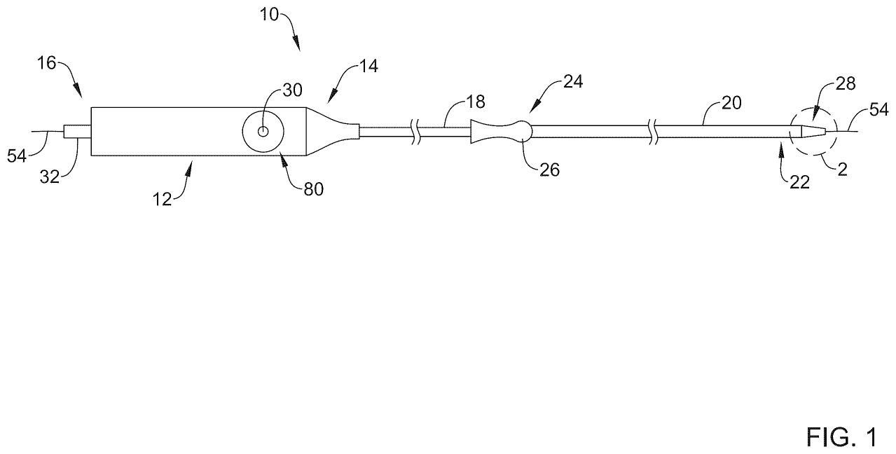

FIG. 1 illustrates an example system 10 for establishing a drainage pathway and deploying a drainage stent between a first body organ (e.g., stomach) and a body lumen or cavity (e.g., biliary duct, hepatic duct, bile duct, etc.). In at least some instances, the system 10 is configured to be used with an endoscope (e.g., and/or with an endoscopic ultrasound device) for performing an endoscopic ultrasound-guided hepaticogastrostomy or other similar medical procedure. As will be described in more detail herein, the system 10 may be advanced through at least a portion of the digestive tract (e.g., esophagus) of a patient, used to pierce/puncture through a wall of the stomach and into a lumen/duct along the biliary tract, whereby a covered drainage stent may then be deployed to connect the duct (e.g., biliary duct, hepatic duct, bile duct, etc.) to the stomach cavity.

The system 10 may include a handle or handle assembly 12. The handle 12 may include a distal end region 14 and a proximal end region 16. As illustrated in FIG. 1, the distal end region 14 may be coupled to the proximal end region of an inner shaft 18. Further, the inner shaft 18 may extend through a lumen of an outer shaft 20.

Additionally, FIG. 1 illustrates that the distal end of the inner shaft 18 may be attached to a tip member 28. In some examples, the tip member 28 may include a marker configured to help a user guide the tip member 28 during a medical procedure. It can be appreciated that the marker positioned on the tip member 28 may be formed from a band or ring of solid radiopaque metal, such as gold, tungsten, tantalum, platinum other similar materials.

FIG. 1 further illustrates that the outer shaft 20 may include a distal end region 22 and a proximal end region 24. FIG. 1 illustrates that, in some examples, a distal end portion of the tip member 28 may be positioned adjacent to the distal end of the outer shaft 20. In other words, the tip member 28 may include a proximal face which contacts and/or abuts a distal face of the outer shaft 20.

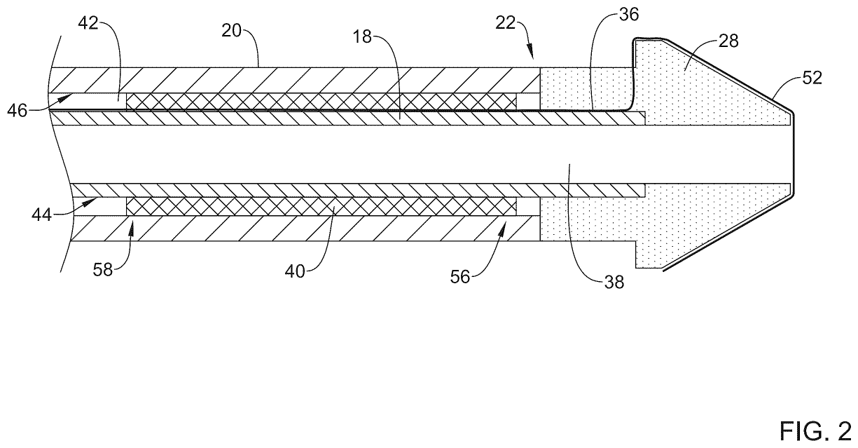

FIG. 1 further illustrates that the proximal end region 24 of the outer shaft may include grip member 26. The grip member 26 may be configured to include an ergonomic shape which permits a user to easily and comfortably grasp the grip member 26. As will be discussed in greater detail below, the grip member 26 may permit a user to actuate the outer shaft 20 relative to the inner shaft 18 and the handle 12. For example, the system 10 may be configured to permit a user to grasp the handle 12 with one hand while using their opposite hand to grasp and pull the grip member 26 toward the handle 12, thereby translating the outer shaft 20 relative to the inner shaft 18. As will be discussed in greater detail herein, the distal-to-proximal translation of the outer shaft 20 relative to the inner shaft 18 may deploy a stent 40 (shown in FIG. 2) positioned between the outer shaft 20 and the inner shaft 18.

As will be discussed in greater detail herein, the system 10 may further include one or more components configured to delivery an electrical current to and electrical cautery element positioned on the tip member 28. FIG. 1 illustrates that the handle 12 may include an electrical pin connector 30 positioned along the distal end region 14. FIG. 1 illustrates that the electrical pin connector 30 may be positioned within an electrosurgical cable attachment element 80.

The electrical pin connector 30 may be coupled to a console via and electrical cable configured to be attached to the electrosurgical cable attachment element 80. A cable attached to the electrosurgical cable attachment element 80 may include an electrical wire configured to connect with the electrical pin connector 30 and thereby provide an electrical current (e.g., electrical energy) to the system 10. It can be further appreciated that the handle 12 may be coupled to one or more elements configured to permit a user to control the timing and amount of electrical energy delivered to the system 10 via the electrical pin connector 30. For example, the handle 12 may be coupled to a foot pedal which permits a user to control the timing and amount of electrical energy delivered to the system 10 via the electrical pin connector 30. It is further contemplated that, in some examples, the electrical pin connector 30 may be positioned on a proximal end region 16 or on any portion of the handle 12 between the proximal end region 16 and the distal end region 14.

FIG. 1 further illustrates that the proximal end region 16 of the handle 12 may include a port 32 (e.g., luer fitting, etc.). The port 32 may be configured to permit the attachment of a syringe, Y-adaptor, etc. thereto. It can be appreciated that the port 32 may permit the injection of fluid (e.g., contrast, saline, etc.) into a lumen of the inner shaft 18. Further, it can be appreciated that port 32 may permit aspiration of fluid through the lumen of the inner shaft 18. Further yet, it can be appreciated that the port 32 may permit a guidewire 54 to be inserted and extend within the lumen 38 (shown in FIG. 2) of the inner shaft 18.

FIG. 2 illustrates a distal portion of the system 10 of FIG. 1. FIG. 2 illustrates the distal end 22 of the outer shaft 20 positioned adjacent to the proximal end of the tip member 28. Further, FIG. 2 illustrates the inner shaft 18 extending within the lumen of the outer shaft 20 whereby the distal end region of the inner shaft 18 is coupled to the tip member 28.

FIG. 2 further illustrates that the system 10 may include an electrical wire 36 extending along the length of the inner shaft 18. In some examples, the electrical wire 36 may extend along an outer surface 44 of the inner shaft 18. In other examples, the electrical wire 36 may extend within a channel or lumen of the inner shaft 18. For example, the inner shaft 18 may be constructed as a multi-lumen shaft whereby the electrical wire 18 extends within a lumen of the multi-lumen inner shaft 18.

FIG. 2 further illustrates that the electrical wire 36 may extend within a channel or passageway within the tip member 28 to a position in which the electrical wire 36 couples to an electrical contact surface 52 positioned on a distal-facing surface of the tip member 28. It can be appreciated that the electrical wire 36 may extend along the inner shaft 18 from a proximal end thereof, through the tip member 28 to the electrical contact surface 52.

It can be further appreciated the distal end of the electrical wire 36 may be attached to the electrical pin connector 30 (shown in FIG. 1). It can be appreciated that the electrical wire 36 may be configured to transfer electrical energy from the electrical pin connector go the electrical contact surface 52. It can be further appreciated that energizing the electrical contact surface 52 may permit the electrical contact surface to cauterize and cut through tissue. It can be appreciated that the electrical contact surface 52 may extend circumferentially around the outer surface of the tip member 28. It can be further appreciated that the electrical contact surface 52 may include an aperture to permit a guidewire to extend therethrough.

FIG. 2 further illustrates that the system 10 may further include a stent 40 positioned in a gap 42 formed between the outer surface 44 of the inner shaft and the inner surface of the outer shaft 20. The stent 40 may be a self-expandable stent configured to expand from a first constrained configuration to a second expanded configuration. In some examples, the system 10 may be configured to deploy stents that are about 10 mm to about 110 mm long, or about 20 mm to about 100 mm long, or about 30 mm to about 90 mm long, or about 40 mm to about 80 mm long, or about 40 mm to about 70 mm long, or about 50 mm to about 100 mm long.

In some examples, the stent 40 may include a covering and/or coating positioned along a portion or the entire length of the stent 40. Further, in some examples, the distal end region 56, the proximal end region 58 or both the distal and proximal end regions 56, 58 of the stent 40 may include a flared portion. It can be appreciated that the system 10 shown in FIG. 2 shows the stent 40 positioned in a pre-deployed configuration in which the outer shaft 20 extends over the entire length of the stent 40, thereby maintaining the stent 40 in a constrained, pre-deployed configuration.

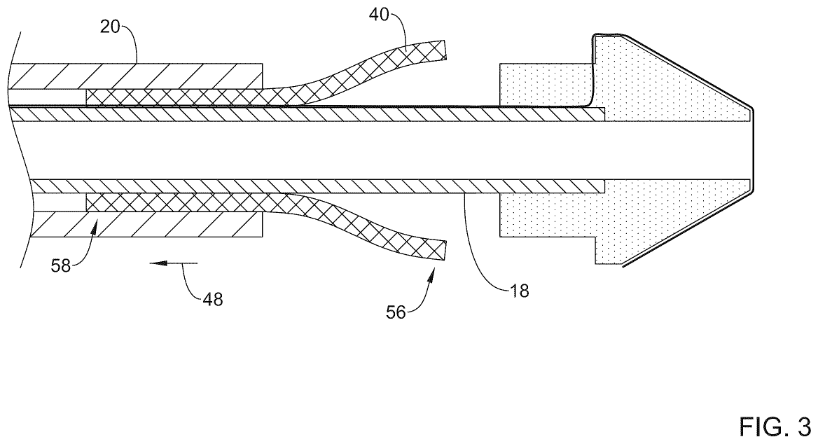

FIG. 3 illustrates that the distal-to-proximal retraction (depicted by reference numeral 48) of the outer shaft 20 relative to the inner shaft 18 and the stent 40. As described herein, distal-to-proximal retraction of the outer shaft 20 relative to the inner shaft 18 and the stent 40 may be accomplished via a user grasping the handle 12 with one hand while using their opposite hand to grasp and pull the grip member 26 (shown in FIG. 1) toward the handle 12, thereby translating the outer shaft 20 relative to the inner shaft 18. FIG. 3 further illustrates that the distal-to-proximal retraction of the outer shaft 20 relative to the inner shaft 18 exposes the stent 40, thereby permitting the stent 40 to shift from a constrained configuration to an expanded configuration. It can be appreciated that the distal-to-proximal retraction of the outer shaft 20 may progressively uncover the stent 40 from distal end region 56 to the proximal end region 58.

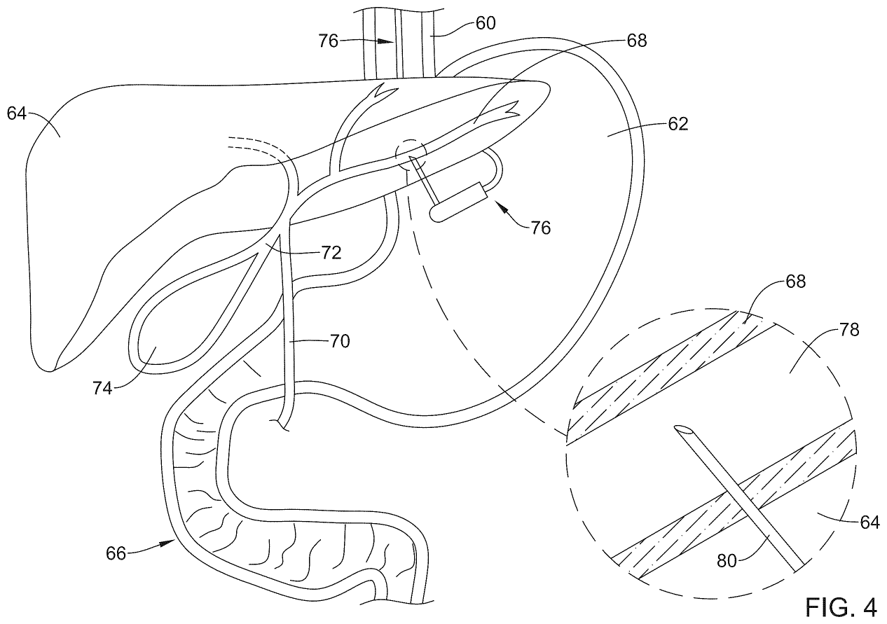

FIG. 4 illustrates an overview of the biliary system or tree. FIG. 4 illustrates the esophagus 60 connected to the stomach 62 (note that a portion of the liver 64 is masking the opening in which the esophagus 60 empties into the gastric lumen of the stomach 62). A portion of the duodenum 66 is shown extending from a portion of the stomach 62. Further, the hepatic ducts, denoted by the reference numeral 68, are connected to the liver 64 and empty into the bile duct 70. Similarly, the cystic duct 72, being connected to the gall bladder 74, also empties into the bile duct 70.

As discussed herein, an endoscopic or biliary procedure may include advancing a medical device through the esophagus 60 to a suitable location within the stomach 62, whereby the medical device may be utilized to access a suitable location along the biliary tree and then performing the appropriate intervention. For example, FIG. 4 illustrates an endoscope 76 extending through the esophagus 60 to a suitable location within the stomach 62. The disclosure is not intended to be limited to disposing the endoscope 76 in the stomach 62 as other locations are contemplated including other portions of the digestive system including the stomach 62. For example, the system 10 and methods utilizing the system 10 may include draining the biliary tree between the bile duct 70 and the duodenum 66.

The detailed view of FIG. 4 further illustrates an initial step in forming a fistula extending from the gastric lumen of the stomach 62, through the tissue of the liver 64 and into the lumen 78 of a duct 68 (e.g., biliary duct, hepatic duct, bile duct, etc.). For example, FIG. 4 illustrates an access needle 80 exiting out of the endoscope 76 and into the liver 64. Further, the detailed view of FIG. 4 illustrates the distal end of the access needle 80 extending through the liver 64 and into the lumen 78 of the duct 68 (e.g., biliary duct, hepatic duct, bile duct, etc.). Additionally, in some examples, the initial step in forming the fistula extending from the gastric lumen of the stomach 62, through the tissue of the liver 64 and into the lumen 78 of a duct 68 (e.g., biliary duct, hepatic duct, bile duct, etc.) may be formed with a guidewire having a cautery tip in lieu of an access needle described herein. A guidewire having a cautery tip may extend within the lumen 38 of the inner member 18.

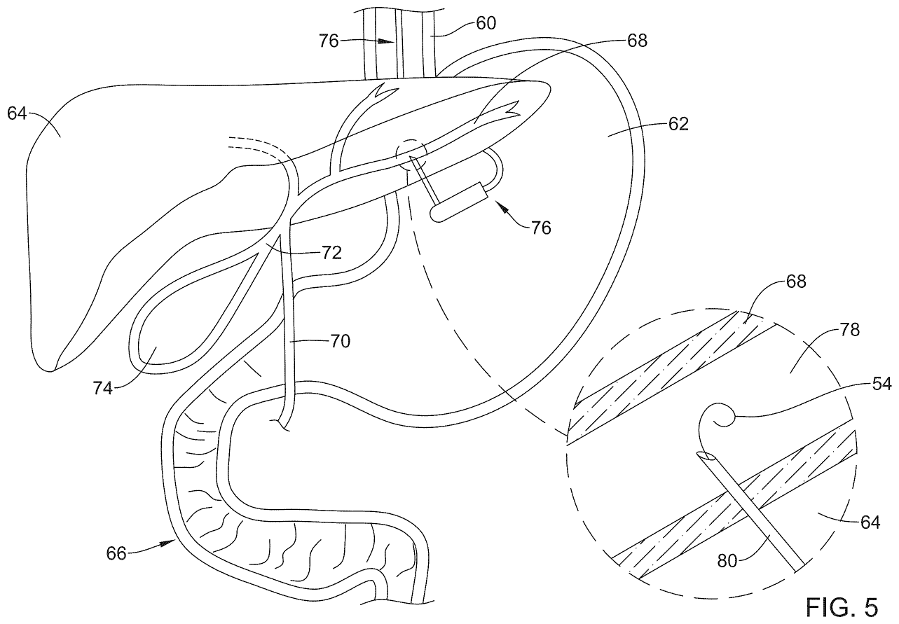

FIG. 5 illustrates another step in forming a fistula extending from the gastric lumen of the stomach 62, through the tissue of the liver 64 and into the lumen 78 of the duct 68 (e.g., biliary duct, hepatic duct, bile duct, etc.). FIG. 5 illustrates that the guidewire 54 (also shown in FIG. 1) may be tracked through the endoscope 76 and the access needle 80 to a position in which the distal end region of the guidewire is positioned in the lumen 78 of the duct 68 (e.g., biliary duct, hepatic duct, bile duct, etc.). It can be appreciated the after the distal end region of the guidewire 78 is positioned in the duct 68 (e.g., biliary duct, hepatic duct, bile duct, etc.), the access needle 80 may be removed from the endoscope 76.

As discussed herein, the system 10 may be used to form a fistula extending from the gastric lumen of the stomach 62, through the tissue of the liver 64 and into the lumen 78 of the duct 68 (e.g., biliary duct, hepatic duct, bile duct, etc.). It can be appreciated that a portion of the system 10 may extend through a channel (e.g., a working channel) of the endoscope 76, whereby the handle assembly 12 and grip member 26 may be manipulated to deploy the stent 40 across the pathway formed between gastric lumen of the stomach 62 and the lumen 78 of the duct 68 (e.g., biliary duct, hepatic duct, bile duct, etc.).

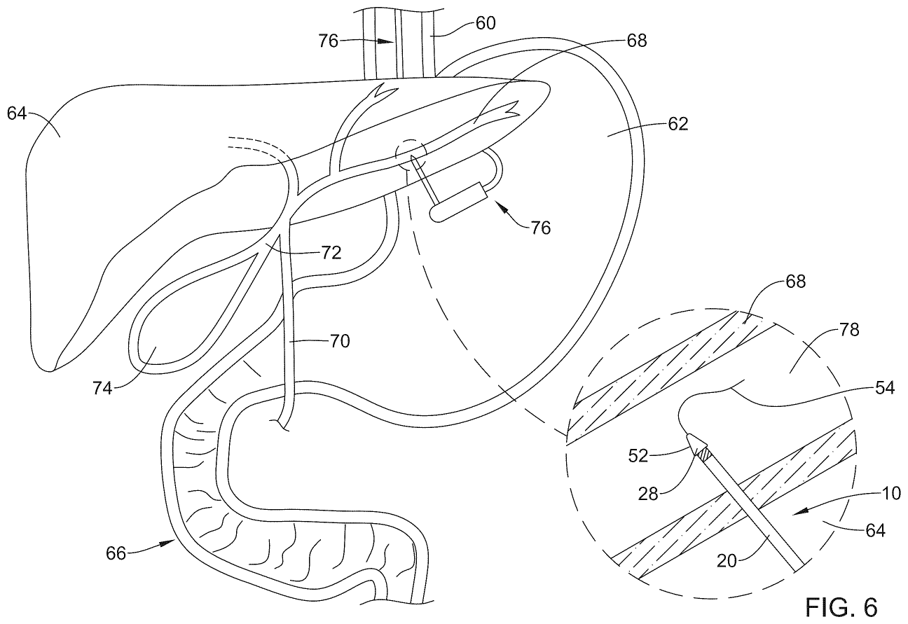

For example, FIG. 6 illustrates that the distal end portion of the system 10 has been tracked over the guidewire 54 and through a channel of the endoscope 76 whereby the tip member 28 exits the distal end of the endoscope 76 within the stomach 62. Further, it can be appreciated from the detail view of FIG. 6 that the electrical contact surface 52 of the tip member 28 may cauterize and thereby form a fistula from the gastric lumen of the stomach 62, through the tissue of the liver 64 and into the lumen 78 of the duct 68 (e.g., biliary duct, hepatic duct, bile duct, etc.).

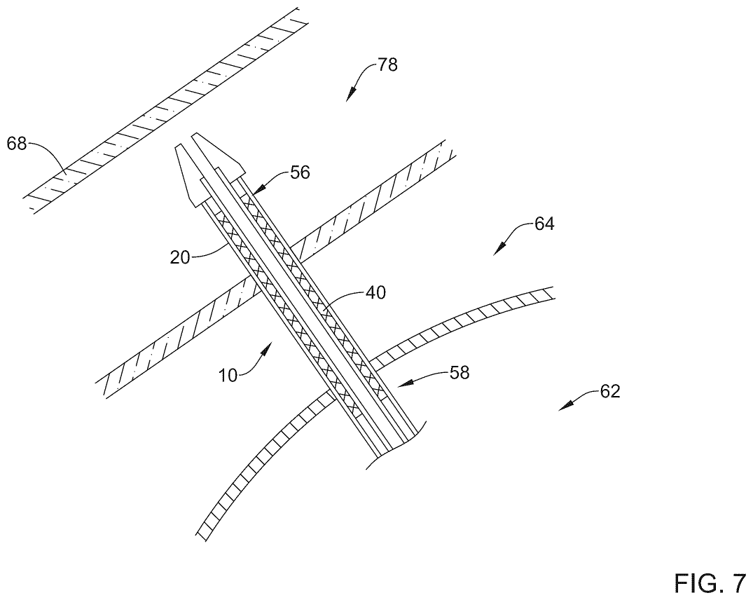

FIGS. 7-9 illustrate the deployment of the stent 40 within the fistula created by the electrical contact surface 52 of the tip member 28. It can be appreciated that the initiation of the deployment of the stent 40 may occur at a time point soon after the fistula is created between the gastric lumen of the stomach 62 and the lumen 78 of the duct 68 (e.g., biliary duct, hepatic duct, bile duct, etc.). Initiating the deployment of the stent 40 at a time point soon after the fistula is created may minimize the risk of body fluid leakage into the tissue surrounding the stomach 62 and duct 68 (e.g., biliary duct, hepatic duct, bile duct, etc.), in addition to minimizing trauma and damage to the tissue surrounding the stomach 62 and duct 68 (e.g., biliary duct, hepatic duct, bile duct, etc.). FIG. 7 illustrates the distal end portion of the system 10 positioned such that the distal end region 56 of the stent 40 is positioned within the lumen 78 of the duct 68 (e.g., biliary duct, hepatic duct, bile duct, etc.) and the proximal end region 58 of the stent 40 is positioned in the gastric lumen of the stomach 62.

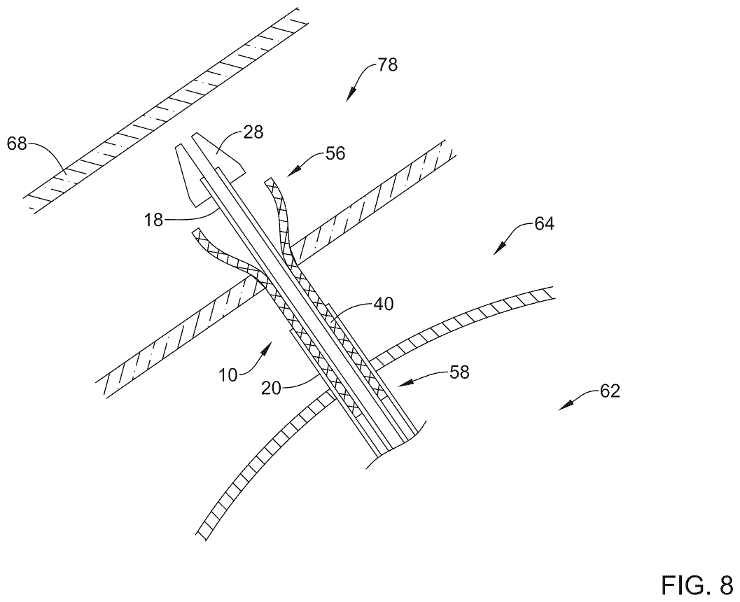

FIG. 8 illustrates the distal-to-proximal retraction of the outer shaft 20 relative to the stent 40, the tip member 28 and the inner shaft 18. As described herein with respect to FIGS. 1-3, the distal-to-proximal retraction of the outer shaft 20 may be accomplished via a user grasping the handle 12 (shown in FIG. 1) with one hand while using their opposite hand to grasp and pull the grip member 26 (shown in FIG. 1) toward the handle 12, thereby translating the outer shaft 20 relative to the inner shaft 18. FIG. 3 further illustrates that the distal-to-proximal retraction of the outer shaft 20 relative to the inner shaft 18 exposes the stent 40, thereby permitting the stent 40 to shift from a constrained configuration to an expanded configuration. It can be appreciated that the distal-to-proximal retraction of the outer shaft 20 may progressively uncover the stent 40 from distal end region 56 to the proximal end region 58. FIG. 8 illustrates the distal end region 56 of the stent 40 expanded within the lumen 78 of the duct 68 (e.g., biliary duct, hepatic duct, bile duct, etc.).

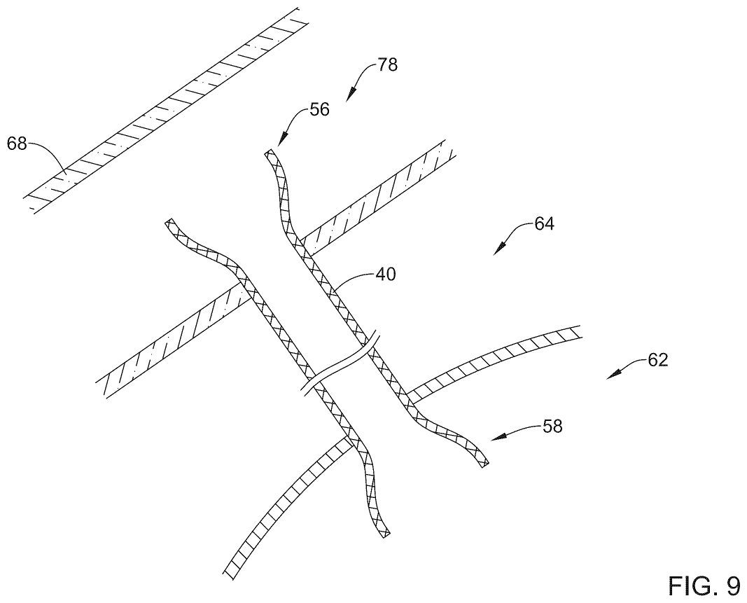

FIG. 9 illustrates the stent 40 after the outer shaft 40 has been proximally retracted to uncover entire length of the stent 40 and thereby permit the proximal end region 58 of the stent 40 to shift from a constrained configuration to an expanded configuration. It can be appreciated that the distal-to-proximal retraction of the outer shaft 20 may progressively uncover the stent 40 from distal end region 56 to the proximal end region 58. FIG. 9 illustrates the proximal end region 58 of the stent 40 expanded within the gastric lumen of the stomach 62. FIG. 9 further illustrates that after the stent 40 has been deployed across the fistula formed between gastric lumen of the stomach 62 and the lumen 78 of the duct 68 (e.g., biliary duct, hepatic duct, bile duct, etc.), the remainder of the system 10 may be retracted through the working channel of the endoscope.

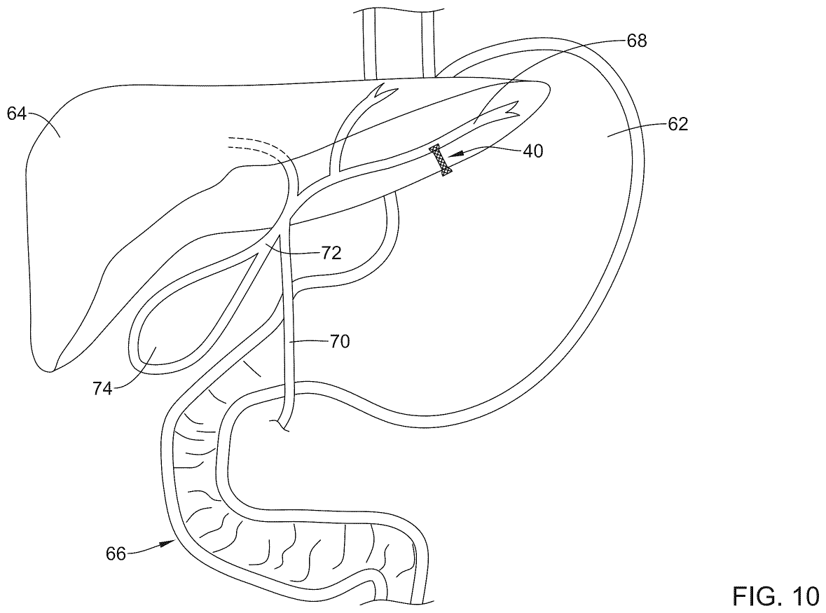

FIG. 10 illustrates the stent 40 extending between the gastric lumen of the stomach 62 and the lumen 78 of the duct 68 (e.g., biliary duct, hepatic duct, bile duct, etc.). It can be appreciated from FIG. 10 that a covered stent 40 positioned as shown in FIG. 10 may permit fluid to drain from the duct 68 (e.g., biliary duct, hepatic duct, bile duct, etc.) into the gastric lumen of the stomach 62.

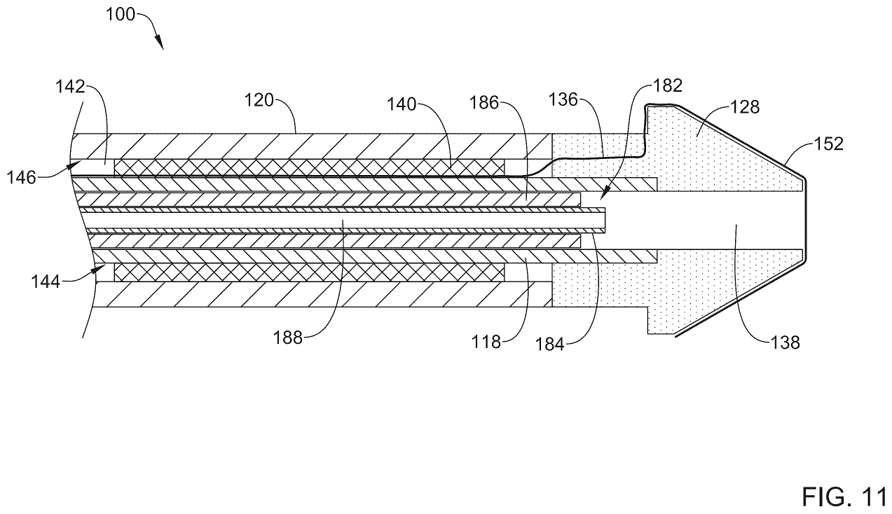

FIG. 11 illustrates the distal end region of another example system 100. The system 100 may be similar in form and function to the system 10 described herein. For example, FIG. 11 illustrates an inner shaft 118 extending within the lumen of an outer shaft 120 whereby the distal end region of the inner shaft 118 is coupled to a tip member 128. Further, FIG. 11 further illustrates that the system 100 may include an electrical wire 136 extending along the length of the inner shaft 118. In some examples, the electrical wire 136 may extend along an outer surface 144 of the inner shaft 118. In other examples, the electrical wire 136 may extend within a channel or lumen of the inner shaft 118. For example, the inner shaft 118 may be constructed as a multi-lumen shaft whereby the electrical wire 118 extends within a lumen of the multi-lumen inner shaft 118.

FIG. 11 further illustrates that the electrical wire 136 may extend within a channel or passageway within the tip member 128 to a position in which the electrical wire 136 couples to an electrical contact surface 152 positioned on a distal-facing surface of the tip member 128. It can be appreciated that the electrical wire 136 may extend along the inner shaft 118 from a proximal end thereof, through the tip member 128 to the electrical contact surface 152.

It can be further appreciated the distal end of the electrical wire 136 may be attached to the electrical pin connector 30 (shown in FIG. 1). It can be appreciated that the electrical wire 136 may be configured to transfer electrical energy from the electrical pin connector go the electrical contact surface 152. It can be further appreciated that energizing the electrical contact surface 152 may permit the electrical contact surface to cauterize and cut through tissue. It can be appreciated that the electrical contact surface 152 may extend circumferentially around the outer surface of the tip member 128. It can be further appreciated that the electrical contact surface 152 may include an aperture to permit a guidewire to extend therethrough.

Additionally, FIG. 11 illustrates that the system 100 may further include a stent 140 positioned in a gap 142 formed between the outer surface 144 of the inner shaft and the inner surface of the outer shaft 120. The stent 140 may be similar in form and function to the stent 40 described herein. For example, the stent 140 may be a self-expandable stent configured to expand from a first constrained configuration to a second expanded configuration. In some examples, the system 10 may be configured to deploy stents that are about 10 mm to about 110 mm long, or about 20 mm to about 100 mm long, or about 30 mm to about 90 mm long, or about 40 mm to about 80 mm long, or about 40 mm to about 70 mm long, or about 50 mm to about 100 mm long.

Further, the stent 140 may include a covering and/or coating positioned along a portion or the entire length of the stent 140. Further, in some examples, similar to the stent 40 described herein, the distal end region, the proximal end region or both the distal and proximal end regions of the stent 140 may include a flared portion. It can be appreciated that the system 100 shown in FIG. 11 shows the stent 140 positioned in a pre-deployed configuration in which the outer shaft 120 extends over the entire length of the stent 140, thereby maintaining the stent 140 in a constrained, pre-deployed configuration.

FIG. 11 further illustrates that the system 100 may include a cannula assembly 182 which is positioned within the lumen 138 of the inner shaft 118. The cannula assembly 182 may include an electrode 184 and an insulation member 186. It can be appreciated that the insulation member 186 may substantially surround all or a portion of the electrode 184. The insulation member 186 may be fixedly attached to the outer surface of the inner shaft 118. In other examples, the insulation member 186 may not be fixedly attached to the outer surface of the inner shaft 118. In some examples, such as that illustrated in FIG. 11, the distal end of the electrode 184 may extend beyond the distal end of the insulation member 186.

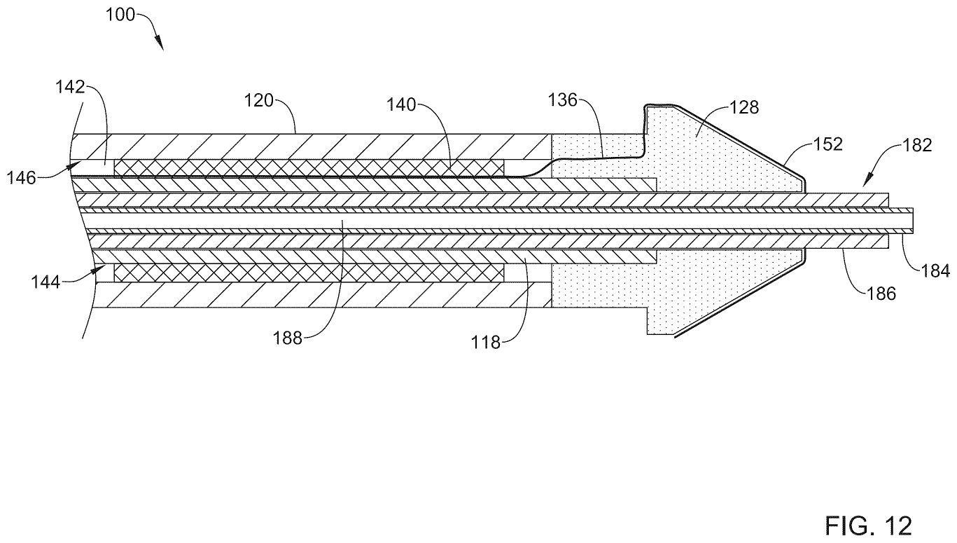

FIG. 12 illustrates that the cannula assembly 182 (including both the electrode 184 and the insulation member 186) may be configured to translate relative to the outer shaft 120, the inner shaft 118, the stent 140 and/or the tip member 128. It can be appreciated that, in some examples, a user may be able to actuate the cannula assembly 182 from a position outside the patient. For example, the handle 12 (shown in FIG. 1) may include one or more actuation members which permit a user to actuate the cannula assembly 182 from a first position in which the cannula assembly 182 is positioned within the lumen 138 of the inner shaft 118 to a second position in which the cannula assembly 182 is positioned distal of the distal end of the tip member 128.

It can be appreciated that translating the cannula assembly 182 to a position in which the cannula assembly 182 is positioned distal of the distal end of the tip member 128 may permit the electrode 184 to contact tissue. Accordingly, it can be further appreciated that the electrode 184 may be configured to cauterize and form a pathway in tissue. For example, the electrode 184 may be configured to cauterize and form a pathway from the gastric lumen of the stomach 62, through the tissue of the liver 64 and into the lumen 78 of the duct 68 (e.g., biliary duct, hepatic duct, bile duct, etc.).

It can be appreciated that the system 100 may be configured to perform the methodology described with respect to FIGS. 4-10 to form a pathway from the gastric lumen of the stomach 62, through the tissue of the liver 64 and into the lumen 78 of the duct 68 (e.g., biliary duct, hepatic duct, bile duct, etc.). However, it can be appreciated that the system 100 may not utilized an access needle 80 (shown in FIG. 4) to form an initial pathway for a guidewire 54 (shown in FIG. 5). Rather, it can be appreciated that the electrode 184 may be configured to form the initial pathway for a guidewire to extend from the gastric lumen of the stomach 62, through the tissue of the liver 64 and into the lumen 78 of the duct 68 (e.g., biliary duct, hepatic duct, bile duct, etc.). After the initial pathway is created via the electrode 184, a guidewire may be inserted through the lumen 188 of the electrode 184 from the gastric lumen of the stomach 62, through the tissue of the liver 64 and into the lumen 78 of the duct 68 (e.g., biliary duct, hepatic duct, bile duct, etc.). After the guidewire is positioned, a user may advance the system 100 into position such that the distal end of the stent 140 is positioned in the duct 68 (e.g., biliary duct, hepatic duct, bile duct, etc.) and the proximal end of the stent 140 is positioned in the stomach 62, as described herein with respect to FIGS. 7-10.

Similar to that described with respect to deployment of the stent 40 shown in FIGS. 7-10, the initiation of the deployment of the stent 140 may occur at a time point soon after the pathway is created between the gastric lumen of the stomach 62 and the lumen 78 of the duct 68 (e.g., biliary duct, hepatic duct, bile duct, etc.). Initiating the deployment of the stent 140 at a time point soon after the pathway is created by the electrical contact surface 152 may minimize the risk of body fluid leakage into tissue surrounding the stomach 62 and duct 68 (e.g., biliary duct, hepatic duct, bile duct, etc.), in addition to minimizing trauma and damage to the tissue surrounding the stomach 62 and duct 68 (e.g., biliary duct, hepatic duct, bile duct, etc.).

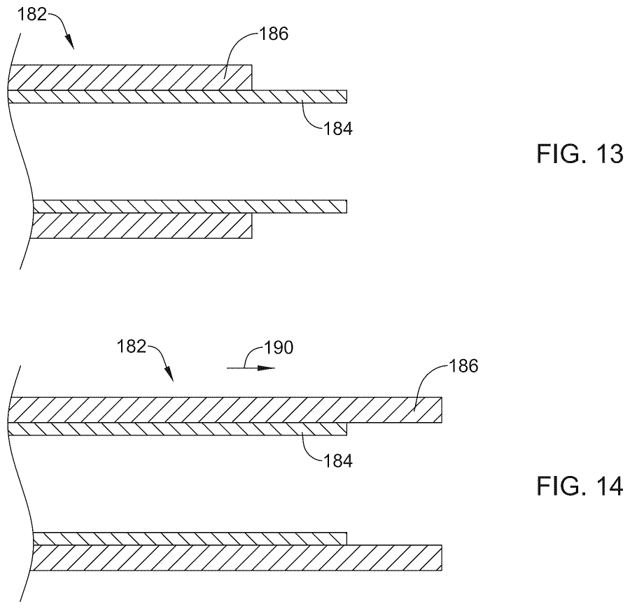

FIGS. 13-14 illustrate that, in some examples, the insulation member 186 may be configured to translate relative to the electrode 184. For example, FIG. 13 illustrates that cannula assembly 182 in a configuration like that described with respect to FIGS. 11-12, whereby the distal end of the electrode 184 extends beyond the distal end of the insulation member 186. As described herein, exposing the distal end of the electrode 184 may permit the electrode 184 to cauterize and form a pathway from the gastric lumen of the stomach 62, through the tissue of the liver 64 and into the lumen 78 of the duct 68 (e.g., biliary duct, hepatic duct, bile duct, etc.). However, prior to passing the guidewire through the lumen 188 of the electrode 184, FIG. 14 illustrates that the distal end of the insulation member 186 may be translated (depicted by reference numeral 190) in a proximal-to-distal direction such that it extends beyond and covers the distal end of the electrode 184. It can be appreciated that the portion of the insulation member 186 covering the distal end of the electrode 184 may protect a guidewire from engaging and being damaged when extending through distal end of the electrode 184.

Further, after the insulation member 186 is translated and covers the distal end of the electrode 184, a guidewire may be inserted through the lumen 188 of the electrode 184 from the gastric lumen of the stomach 62, through the tissue of the liver 64 and into the lumen 78 of the duct 68 (e.g., biliary duct, hepatic duct, bile duct, etc.). After the guidewire is positioned, a user may advance the system 100 into position such that the distal end of the stent 140 is positioned in the duct 68 (e.g., biliary duct, hepatic duct, bile duct, etc.) and the proximal end of the stent 140 is positioned in the stomach 62, as described herein with respect to FIGS. 7-10.

Additionally, it can be appreciated that while the systems 10, 100 are described herein as “over-the-wire” delivery systems, any of the systems 10, 100 and configurations thereof may be configured as a “rapid exchange” (e.g., single operator exchange) system.

The materials that can be used for the various components of the system 10 may include those commonly associated with medical devices. For simplicity purposes, the following discussion makes reference to the various components of the system 10.

The various components of the system 10 may be made from or otherwise includes a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304 L, and 316 LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A system for positioning a stent within a tissue pathway, the system comprising:
a handle;
an inner shaft having a lumen, a proximal end coupled to the handle and a distal end coupled to a tip member;
an outer shaft having a proximal end region and a lumen, wherein at least a portion of the inner shaft extends within the lumen of the outer shaft;
a grip member coupled to the proximal end region of the outer shaft;
a stent positioned between an inner surface of the outer shaft and an outer surface of the inner shaft;
an electrically conductive element coupled to the tip member;
an electrical wire having a proximal end coupled to the handle and a distal end coupled to the electrically conductive element; and
a cannula assembly configured to be positioned within the lumen of the inner shaft, wherein the cannula assembly includes an electrode and an insulation member.

2. The system of claim 1, wherein the cannula assembly is configured to translate relative to the tip member.

3. The system of claim 2, wherein the tip member includes a lumen in fluid communication with the lumen of the inner shaft, and wherein the cannula assembly is configured to translate within both the lumen of the inner shaft and the lumen of the tip member.

4. The system of claim 3, wherein the cannula assembly is configured to translate relative to the lumen of the inner shaft and the lumen of the tip member such that the electrode is positioned distal of the distal end of the tip member.

5. The system of claim 4, wherein the electrode is configured to cauterize tissue when positioned distal of the distal end of the tip member.

6. The system of claim 5, wherein the electrode is configured to be retracted into the lumen of the tip member, the lumen of the inner shaft or both the lumen of the tip member and the lumen of the inner shaft after cauterizing tissue.

7. The system of claim 6, wherein the electrode includes a lumen, and wherein the electrode lumen is configured to permit a guidewire to extend therein.

8. The system of claim 6, wherein the electrically conductive element is configured to cauterize tissue along a tissue pathway formed by the electrode.

9. The system of claim 1, wherein the handle includes an actuation member configured to translate the cannula assembly within the lumen of the inner shaft.

10. The system of claim 1, wherein the insulation member is fixedly attached to the electrode.

11. The system of claim 1, wherein the insulation member is slidable relative to the electrode.

12. The system of claim 11, wherein the insulation member is configured to shift between a first position in which a distal end of the insulation member is positioned proximal to the distal end of the electrode to a second position in which the distal end of the insulation member is positioned distal to the distal end of the electrode.

13. The system of claim 12, wherein the insulation member covers the distal end of the electrode in the second configuration.

14. The system of claim 13, wherein a guidewire is configured to translate through a lumen of the electrode when the insulation member is covering the distal end of the electrode.

15. A system for positioning a drainage stent between a biliary duct and the stomach, the system comprising:
a handle;
an inner shaft having a lumen, a proximal end coupled to the handle and a distal end coupled to a tip member;
an outer shaft having a proximal end region and a lumen, wherein at least a portion of the inner shaft extends within the lumen of the outer shaft;
a grip member coupled to the proximal end region of the outer shaft;
a stent positioned between an inner surface of the outer shaft and an outer surface of the inner shaft;
an electrically conductive element coupled to the tip member; and
an electrical wire having a proximal end coupled to the handle and a distal end coupled to the electrically conductive element;
wherein the electrically conductive element is configured to cauterize a tissue pathway between the biliary duct and the stomach such that the stent can be deployed within a portion of the tissue pathway contiguous in time with forming the tissue pathway in order to reduce leakage.

16. The system of claim 15, further comprising a cannula assembly configured to translate relative to the tip member.

17. The system of claim 16, wherein the tip member includes a lumen in fluid communication with the lumen of the inner shaft, and wherein the cannula assembly is configured to translate within both the lumen of the inner shaft and the lumen of the tip member.

18. The system of claim 17, wherein the cannula assembly is configured to translate within both the lumen of the inner shaft and the lumen of the tip member such that the electrode is positioned distal of the distal end of the tip member.

19. The system of claim 18, wherein the electrode is configured cauterize tissue when positioned distal of the distal end of the tip member.

20. A method for positioning a drainage stent between a biliary duct and the stomach, the method comprising:
positioning a stent delivery system adjacent a wall of the stomach, the stent delivery system including:
a handle;
an inner shaft having a lumen, a proximal end coupled to the handle and a distal end coupled to a tip member;
an outer shaft having a proximal end region and a lumen, wherein at least a portion of the inner shaft extends within the lumen of the outer shaft;
a grip member coupled to the proximal end region of the outer shaft;
a stent positioned between an inner surface of the outer shaft and an outer surface of the inner shaft;
an electrically conductive element coupled to the tip member; and
an electrical wire having a proximal end coupled to the handle and a distal end coupled to the electrically conductive element;
a cannula assembly configured to be positioned within the lumen of the inner shaft, wherein the cannula assembly includes an electrode and an insulation member;
advancing the cannula assembly within the lumen of the inner shaft to a position in which a distal end of the electrode is distal to the tip member;
cauterizing tissue with the electrode to form a pathway between the biliary duct and the stomach;
retracting the cannula assembly into the lumen of the inner shaft.
